(19) United States
(12) Patent Application Publication
(10) Pub. No.: US 2015/0313967 A1
(43) Pub. Date: Nov. 5, 2015

(54) Methods and Agents for Wound Healing

(71) Applicants: Mei CHEN, Altadena, CA (US); David WOODLEY, Altadena, CA (US)

(72) Inventors: Mei CHEN, Altadena, CA (US); David WOODLEY, Altadena, CA (US)

(73) Assignee: UNIVERSITY OF SOUTHERN CALIFORNIA, Los Angeles, CA (US)

(21) Appl. No.: 14/736,110

(22) Filed: Jun. 10, 2015

Related U.S. Application Data
(63) Continuation of application No. 13/610,581, filed on Sep. 11, 2012.
Application No. 13/610,581 is a continuation of application No. PCT/US2011/028227, filed on Mar. 11, 2011.
(60) Provisional application No. 61/313,034, filed on Mar. 11, 2010.

(51) Int. Cl. A61K 38/39; A61K 9/00; A61K 38/18

(52) U.S. Cl. CPC A61K 38/39; A61K 38/1858; A61K 9/0014

(57) ABSTRACT

This invention provides compositions and methods for topically treating skin wounds. The composition comprises C7, C7M, a variant thereof, or a combination thereof in a pharmaceutically acceptable carrier. The method comprises the steps of topically applying compositions of this invention to the skin wound.

### CROSS-REFERENCE TO RELATED APPLICATIONS

This application is a continuation of U.S. application Ser. No. 13/610,581, filed on Sep. 11, 2012, which is a continuation of PCT/US2011/028227, filed on Mar. 11, 2011, which claims the benefit of U.S. Provisional Application No. 61/313,034, filed on Mar. 11, 2010. The above applications are hereby incorporated herein by reference.

### FIELD OF THE INVENTION

The invention pertains to the field of skin wound healing. More particularly, the invention pertains to topical application of a composition comprising collagen type VII as a medicament for healing of skin wounds.

### BACKGROUND OF THE INVENTION

Healing wounds in human skin is a major medical problem, particularly in the elderly patient population. According to the Wound Healing Society, about 15% of older adults suffer from chronic, hard-to-heal wounds [1]. It is also estimated that about 18% of diabetic patients over the age of 65 years will have chronic, non-healing skin ulcers [2]. To improve the healing process, researchers have been considering topically applying epidermal growth factor as a promising therapy. This therapy has been shown to accelerate wound closure of acute wounds in patients [3-5]. However, due to the high cost and other practical considerations, this strategy has not been commercially viable as a general solution for wound healing. So far, only platelet-derived growth factor has been approved by the Federal Drug Administration for treatment of non-healing diabetic foot ulcers. Even with this therapy, practitioners have found it to be limiting and not always successful. One difficulty associated with the topical application of growth factors is that the wound bed is often laden with proteolytic enzymes which tend to degrade and nullify the applied agent.

To investigate better approaches of skin wound healing, various models of skin diseases have been used. In particular, a genetically inherited skin disease known as Dystrophic forms of Epidermolysis Bullosa (DEB) in children has provided valuable insights.

DEB is an incurable genetic disease caused by a gene defect in the gene that encodes for type VII collagen. Children who suffer from DEB are born with skin fragility, blistering, and repeated wounding and healing of their skin wounds [6]. In these children, their wounds will typically heal with fibrosis, scarring, and small epidermal inclusion cysts called milia. Because the outer epidermal layer of the DEB patient adheres poorly to the underlying dermal connective tissue, even the slightest trauma will cause epidermal-dermal disadherence. Therefore, DEB patients suffer from chronic skin wounds. Studies have found that the poor skin adherence is due to a defect in the gene COL7A1 which encodes for type VII (anchoring fibril) collagen (C7); a protein that serves to anchor the epidermis onto the dermis [7,8].

At the molecular level, C7 is composed of three identical alpha chains, each consisting of a 145-kDa central collagenous triple-helical segment, flanked by a large 145-kDa amino-terminal, non-collagenous domain (NC1), and a small 34-kDa carboxyl-terminal non-collagenous domain (NC2) [9,10].

Within the extracellular space, C7 molecules form antiparallel dimers which aggregate laterally to form anchoring fibrils. In normal skin, C7 forms anchoring fibrils ranging from about 200-700 nm in size that emanate from epidermal-dermal junction (EDJ) and extend perpendicularly down into the papillary dermis. In DEB patients, the EDJ is characterized by a paucity of normal anchoring fibrils. Based on the underlying etiology of the disease, one logical approach for treating the disease is to correct the genetic defect through gene therapy.

There are several recent studies related to ex vivo gene therapy for DEB. In the study by Oritz-Urda et al., COL7A1 cDNA was successfully and stably integrated into C7-null keratinocytes from recessive DEB (RDEB) patients using a phi C31 integrase-based non-viral gene transfer approach. By transplanting a human skin equivalent comprising these gene-corrected cells onto severe combined immunodeficient (SCID) mice, they showed that many of the RDEB features were corrected after gene transfer [11].

In another study by the inventors of the present invention, a minimal lentiviral vector was developed to express C7 in RDEB keratinocytes and fibroblasts (in which C7 was absent). This construct was subsequently used to demonstrate the reversion of the RDEB cellular phenotype [12]. In this experiment, the gene-corrected RDEB cells and native un-corrected RDEB cells were used to create a composite human skin equivalent which was then transplanted onto SCID mice. It was shown that the transplanted human skin made with the gene-corrected RDEB cells (but not the control un-corrected RDEB cells) exhibited C7 at the EDJ and the RDEB skin phenotype was corrected. Moreover, in the skin equivalents composed of gene-corrected (but not gene-uncorrected) cells, the transgene-derived C7 also created anchoring fibril structures that were correctly organized into the basement membrane zone (BMZ) lying between the epidermis and dermis.

However, this type of ex vivo approach requires transplantation of gene-corrected cells onto surgically prepared sites of the patient's skin. The experience of using cultured keratinocyte autografts for transplantation onto human wounds had shown that this technology is often fraught with technical difficulties and poor graft take. Therefore, although this ex vivo type of therapy (i.e. gene correcting cells in culture and then transplanting them back as skin equivalents onto the DEB patient) is theoretically possible, the technical hurdles make it in-efficient, logistically difficult, expensive, labor-intensive and of limited efficacy.

As an alternative approach, a more straightforward “direct in vivo gene therapy” was developed. With this approach, DEB wounded skin is directly injected through intradermal injection with gene-corrected RDEB fibroblasts. The gene-corrected intradermally injected cells then set up residence in the DEB skin and synthesize and secrete C7 which is lacking in the DEB skin. Surprisingly, the secreted C7 in the extracellular dermal tissue, binds to the BMZ of the DEB skin and correctly organizes into anchoring fibril structures. Now, the DEB skin which previously lacked C7 and anchoring fibrils, now has these elements and the DEB skin phenotype is corrected. The poor epidermal-dermal adherence is now corrected. This is called “cell therapy” for DEB by the inventors. The inventors also showed that the same events would occur if they intradermally injected full-length or “mini-C7” into DEB skin, the injected C7 would bind to the BMZ of the DEB skin and form correctly-organized anchoring fibrils and correct the DEB skin phenotype. The inventors called this “protein therapy” for DEB.

Full-length C7 contains a 39-amino acid interruption in its helical sequence which forms site that is highly susceptible to degradation by protease. The C7M, hereinafter also referred to as C7M, was designed by the inventors to contain the intact noncollagenous domains, NC1 and NC2, and half of the central collagenous domain. By excluding the 39 amino acid interrupt, this C7M is made highly stable. The enhanced stability of C7M over native C7 may better withstand proteolytic digestion in RDEB wounds and provide a more sustained gene product in patients. The inventors have shown that the C7M is highly resistant to proteolytic digestion and yet when injected into DEB skin behaves identically to the full-length C7 in that it will bind to the BMZ, create new anchoring fibril structures and correct the DEB skin phenotype. Thirdly, the inventors showed that rather than intradermally injecting RDEB gene-corrected cells or C7 itself into DEB skin, they could inject simply into the DEB skin the lentiviral vector expressing either the full-length C7 or C7M. In this case, the exogenously injected vector was taken up by the endogenous dermal fibroblasts within the dermis of the DEB skin. These endogenous fibroblasts which previously lacked the ability to make C7 or C7M, now could synthesize and secrete these large proteins into the dermal extracellular environment. There, the C7 or C7M homed to the BMZ of the DEB skin, organized into anchoring fibril structures and corrected the DEB skin phenotype. The inventors called this “vector therapy” for DEB [13-16, the relevant portions thereof are incorporated herein by reference].

In recent experiments, the inventors have shown that the intradermal approach is highly efficient if the agents are delivered in the high papillary dermis using a 30 gauge needle with the bevel oriented upward, using a volume between 2 microliters and 2 milliters and injected into four quadrants of DEB skin between 1×1 cm and 6×6 cm.

In a more recent study, the investigators evaluated the feasibility of protein therapy in a C7 null DEB mouse which recapitulates the clinical and ultrastructural features of human RDEB. The investigators intradermally injected purified human C7 (into the new born DEB mice with severe skin blistering and fragility and found that the injected human C7 transported and stably incorporated into the mouse's BMZ and formed anchoring fibrils. The restoration of C7 corrected the DEB murine phenotype as demonstrated by decreased skin fragility and blistering, reduced new blister formation and marked prolonged survival.

Despite the above mentioned advances, there are still no effective methods for treating DEB that is effective and easy to administer. Because patients with severe DEB have widespread lesions and multiple wounds spanning large areas of trauma-prone sites such as the sacrum, hips, feet, mouth, scalp, lower back and hands, the treatment of such DEB patients via any of the three above outlined direct intradermal injection approaches would require numerous injections into multiple wound sites. Accordingly, intradermal injections of the therapeutic agents outlined above (gene-corrected cells, recombinant forms of C7 or C7 expressing vectors) would require site-specific treatment of each and every wound by one or more intradermal injections. While this is doable, such a cumbersome method of treatment still leaves much to be desired. It would be ideal to offer patients with DEB or patients with multiple wounds a single therapy that will require only a single route of delivery but that will “home” to all of the wounds automatically upon delivery to correct the skin wounds located at scattered sites.

Therefore, there still exists a great need for better method of treating skin wounds in general and DEB in particular.

### SUMMARY OF THE INVENTION

In view of the above, it is an object of the present invention to provide a protein therapy for healing skin wounds that is effective and easy to administer.

It is also an object of the present invention to provide a composition that is capable of enhancing wound healing in a subject when applied topically to the subject's skin.

These and other objects of the present invention are satisfied by the unexpected discoveries that topical application of C7 can enhance skin wound closure, improve re-epithelialization and keratinocyte migration. Based on the unexpected discoveries of the invention, various methods and compositions for enhancing wound healing have been devised.

Accordingly, in one aspect, the present invention provides a method for enhancing wound healing in a subject. Embodiments in accordance with this aspect of the invention will generally include the step of administering to the subject an effective amount of a composition containing a skin healing enhancer by applying the composition topically to the subject's skin. The healing enhancer comprises at least one selected from the group consisting of C7, C7M, and a variant thereof.

In a preferred embodiment, the subject is a healthy human. In another preferred embodiment, the subject is one suffering from DEB, more preferably RDEB. Thus, methods in accordance with this embodiment may also be viewed as treatment methods for patients who suffer from DEB, in particular, RDEB. In yet another preferred embodiment, the subject is a diabetic patient.

In another aspect, the present invention also provides a composition for treating DEB. Embodiments in accordance with this aspect of the invention will generally include a skin wound healing enhancer and a pharmaceutically acceptable carrier. In a preferred embodiment, the skin wound healing enhancer is C7, C7M, a variant thereof and a combination thereof. In another alternate preferred embodiment, the composition may further include a secondary skin wound healing enhancer such as PDGF. In a still further embodiment, the composition may further include a protein stabilizing agent such as Hsp90.

Methods and compositions of the present invention will have at least the advantage that they are easy to administer, does not require site-specific application, inexpensive, and effective.

Other aspects and advantages of the invention will be apparent from the following description and the appended claims.

### BRIEF DESCRIPTION OF THE DRAWINGS

FIG. 1 shows the wound healing ability of topical C7 treatment. (A) shows photographs of the wound site. (B) shows a plot of wound size versus time.

FIG. 2 shows images of immunofluorescence staining of mice's skin after topical treatment of C7 at 2 and 4 weeks.

FIG. 3 shows images of immunofluorescence staining of mice's skin after topical treatment of C7 at 2 weeks.

FIG. 4 illustrates that C7 strongly promotes human keratinocyte migration. (A) shows photographs of coverslips plated with different matrix. (B) shows the corresponding migration index.

FIG. 5 illustrates that topical C7-treatment can enhance re-epithelialization of skin wounds. (A) shows composite pictures of H&E staining of wounds with or without topical C7 treatment. (B) shows higher magnification of the wounds.

FIG. 6 illustrates that the truncated domain in C7M plays a role in promoting migration of human keratinocytes. (A) shows a schematics view of C7's domain organization. (B) shows photographs of coverslips plated with various matrices. (C) shows a plot of migration index against each different matrix.

FIGS. 7A-7B show a comparison of wound healing power between topical C7 treatment and topical PDGF treatment.

FIG. 8 shows representative images of C7-treated and vehicle-treated wounds stained with Masson's trichrome.

FIG. 9 shows immunofluorescence staining of the mouse's healed skin.

FIG. 10 shows photographs of wounds on diabetic mice after topical treatment of C7.

### DETAILED DESCRIPTION

### Definitions

Unless otherwise indicated, all terms used herein have the meanings given below, and are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y. and Ausubel F M et al. (1993) Current Protocols in Molecular Biology, John Wiley & Sons, New York, N.Y., for definitions and terms of the art. It is to be understood that this invention is not limited to the particular methodology, protocols, and reagents described, as these may vary.

As used herein, the acronym “C7” stands for collagen type VII encoded by the gene COL7A1.

As used herein, the acronym “C7M” stands for “mini-C7” as described by Chen et al. (J. Biol. Chem. 275(32):24429-24435 (2000), the content of which is incorporated herein by reference.) For the purpose of this invention, “C7M” and “mini-C7” are used interchangeably. Briefly, C7M is formed by selectively removing a portion from the wild-type C7. Wild-type C7 is a protein consisting of 2,944 amino acid residues. It can be further divided into the non-collagenous NC1 domain (residues 1-1253), the central collagenous helical domain (residues 1254-2783), and the carboxyl-terminal NC2 domain (residues 2784-2944). C7M is formed by removing residues 1920-2603 within the central collagenous domain.

As used herein, the term “RDEB” means recessive dystrophic epidermolysis bullosa, which encompasses both Hallopeau-Siemens type RDEB (HS-RDEB) and non-Hallopeau-Siemens type RDEB (non-HS RDEB).

All publications cited herein are expressly incorporated herein by reference for the purpose of describing and disclosing compositions and methodologies that might be used in connection with the invention.

Although the present invention is described in terms of specific exemplary embodiments and examples, it will be appreciated that the embodiments disclosed herein are for illustrative purposes only and various modifications and alterations might be made by those skilled in the art without departing from the spirit and scope of the invention as set forth in the appended claims.

### C7 and C7M Mediated Wound Healing

Skin wounds heal according to a sequence of synchronized events: clot formation, inflammation, production of granulation tissue, fibroplasia, angiogenesis, re-epithelialization, and connective tissue remodeling. Re-epithelialization is critical for closing the open wound, which, in turn, is dependent on keratinocyte migration. Because skin acts as a barrier between the internals of the body and the outside world, speedy healing of open wounds is highly desirable. Slow healing wounds increases the chance of infection and other undesirable conditions that may negatively impact patients' health. There are a number of conditions that can lead to compromised wound healing in a patient. For example, in diabetic patients, clogged arteries may lead to sores and compromised wound healing. Defective wound healing is particularly a devastating problem in patients with DEB, as it can lead to death from aggressive metastatic squamous cell carcinoma.

As mentioned above, patients with dystrophic epidermolysis bullosa (DEB) have incurable skin fragility, blistering and multiple skin wounds due to mutations in the gene that encodes for C7 that holds the epidermal and dermal layers of human skin together. Intradermal injection of gene-corrected DEB fibroblasts, recombinant C7 protein, and lentiviral vectors expressing C7 are all potential therapies for DEB. However, these modalities of applying C7 are either highly complex or very cumbersome to perform.

In this invention, the inventors have unexpectedly discovered that C7 and C7M, when applied topically, are surprisingly effective in healing wounds.

As an example, the inventors first made a 1 square-centimeter full-thickness wound on the back of athymic nude mice and applied 20-40 micrograms of recombinant human C7 in a carrier (e.g. 10% carboxymethylcellulose salt gel) to the skin wounds. Skin biopsies from the wounded areas were obtained every week two weeks after topical application and subjected to immunostaining using an antibody specific for human C7. Surprisingly, the topically applied human C7 stably incorporated into the newly formed BMZ of the mouse's skin.

In contrast, there was no human C7 expression in wounds treated with the carrier alone.

Time course observations and histological analysis revealed that wounds treated with C7, when compared with control wounds (carrier or BSA) demonstrated accelerated wound healing, increased epidermal and dermal regeneration, reduced contraction, and more highly organized collagen fiber deposition, consistent with less scar formation. C7-treated wounds also had increased re-epithelialization due to C7's ability of enhancing keratinocyte migration.

When the healing effect of topical C7 is directly compared to topical recombinant human platelet derived growth factor (PDGF) (0.01% Regranax), a FDA-approved agent that promotes healing of diabetic skin ulcers, the C7-treated wounds demonstrated remarkable enhanced wound closure.

These data demonstrate that topically applied C7 can deliver C7 to the skin BMZ and promote wound healing.

Accordingly, this invention provides composition useful for topically treating skin wounds wherein said composition comprises an effective amount of C7 or C7M in a pharmaceutically suitable carrier.

This invention also provides a novel method for treating skin wounds by topically applying a pharmaceutical composition comprising an effective amount of C7 or C7M in a pharmaceutically acceptable carrier.

### Compositions for Healing Skin Wound

Compositions of the present invention generally include a wound healing enhancer selected from the group consisting of C7, C7M, a variant thereof, and a combination thereof.

For the purpose of the present invention, a variant of C7 or C7M refers to a protein with sequence homology to C7 or C7M of from about 80% to 90%, more preferably from about 90% to 95%. Variations to native C7 sequence and C7M sequence may be made by making single amino acid substitutions, or deletions of non-essential residues such as those within the collagenous triple-helix domain of C7 (residues 1253-2783).

The C7, C7M and their variants can be formulated according to known methods to prepare pharmaceutically useful compositions, whereby these active agents are combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, ordextrins; chelating agents such as EDTA; sugar alcohols such as mannitol or sorbitol; salt-forming counterions such as sodium; and/or nonionic surfactants such as TWEEN™, PLURONICS™ or PEG.

Therapeutic compositions herein generally are placed into a container having a sterile access port, for example, a solution dispensing bag or vial.

The preferred route of administration is by direct topical administration, or by sustained release systems.

Dosages and desired drug concentrations of pharmaceutical compositions of the present invention may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

For topical application, it is preferred to treat patients locally according to wound size, preferably in the range of 1-1000 μg C7/per centimeter square wound. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one patient suffering from one skin disorder, for example, may necessitate delivery in a manner different from that to another patient suffering from a different skin disorder.

Where sustained-release administration of the active ingredients (i.e. C7, C7M, or a variant thereof) is desired in a formulation with release characteristics suitable for the treatment of any disease or disorder requiring administration of the active ingredients, microencapsulation of the active ingredients is contemplated. Microencapsulation of recombinant proteins for sustained release has been successfully performed with human growth hormone (rhGH), interferon-(rhIFN-), interleukin-2, and MN rgp 120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

In a preferred exemplary embodiment, the pharmaceutical carrier is 10% carboxymethylcellulose salt gel, or PBS.

C7 can be obtained by procedures such as described below. For large-scale purification of recombinant C7, serum-free media from gene-corrected RDEB fibroblasts over-expressing C7 or 293 cells stably transfected with a C7 expression construct were equilibrated to 5 mM EDTA, 50 μM PMSF and 50 μM NEM and precipitated with 300 mg/ml ammonium sulfate at 4⁰ C overnight with stifling (4). Precipitated proteins were collected by centrifuging at 1.2×10⁶ g/min for 1 hr, resuspended and dialyzed in Buffer A (65 mM NaCl, 25 mM Tris-HCl, pH 7.8). Following dialysis, insoluble material was collected by centrifugation at 8,600 g for 20 min, and the pellet redissolved in Buffer B (50 mM Tris-HCl pH 7.5, 150 mM NaCl, 5 mM EDTA, 2 mM NEM, 2 mM PMSF). The solution was clarified as above, and the supernatant, S1 was passed over a Q-sepharose column (Pharmacia, Inc., Piscataway, NJ) equilibrated in the same buffer. Elution was then carried out with a linear gradient from 0.2 to 1.0 M NaCl of appropriate volume size. The type VII collagen was eluted at 1 M NaCl.

C7M can be obtained by ammonium sulfate precipitation followed by Q-sepharose chromatograph as described below. For large-scale purification of recombinant C7M, serum-free media from 293 cells stably transfected with an expression construct expressing C7M were equilibrated to 5 mM EDTA, 50 μM PMSF and 50 μM NEM and precipitated with 300 mg/ml ammonium sulfate at 4° C. overnight with stifling (24). Precipitated proteins were collected by centrifuging at 1.2×10⁶ g/min for 1 hr, resuspended and dialyzed in Buffer A (65 mM NaCl, 25 mM Tris-HCl, pH 7.8). Following dialysis, insoluble material was collected by centrifugation at 8,600 g for 20 min, and the pellet redissolved in Buffer B (50 mM Tris-HCl pH 7.5, 150 mM NaCl, 5 mM EDTA, 2 mM NEM, 2 mM PMSF). The solution was clarified as above, and the supernatant, S1, stored at −20° C. The pellet redissolved in Buffer C (50 mM Tris-HCl pH 7.5, 500 mM NaCl, 2 M urea, 5 mM EDTA, 2 mM MEM, and 2 mM PMSF). The solution was clarified as above and the supernatant containing purified minicollagen VII, S2, stored at −20° C.

Variants of C7 and C7M can also be similarly produced.

In a further embodiment, compositions of the present invention may further include a secondary wound healing enhancer such as PDGF.

In a still further embodiment, composition of the present invention may also include a protein stabilization agent such as Hsp90.

### Methods for Healing Skin Wound

Treatment methods in accordance with embodiments of the present invention generally include the step of administering an effective amount of a wound healing composition of the present invention to the skin of a subject in need of the treatment.

Wound healing compositions of the present invention are as described above. The subject to be treated is generally a mammal. Exemplary subjects may include, but not limited to, mice, dogs, horses, pigs, cows, cats, human, and the like.

Compositions of the present invention may be topically applied to a subject's skin by any conventional means including, but not limited to, rubbing, spraying, soaking, etc. Because inventors have unexpected found that C7, C7M, and their variants have the ability to “home” to the wound site, location where the composition is applied is not particularly limited. However, for best results, direct application on the wound site is preferred.

Methods of the present invention are applicable generally to all types of skin wounds, but are particularly effective for certain type of skin disorders involving C7 mutations. In a preferred embodiment, the subject to be treated is a human suffering from DEB, more preferably RDEB. In another preferred embodiment, the subject to be treated is a human suffering from diabetes.

The following examples are provided in order to demonstrate and further illustrate certain embodiments and aspects of the present invention and are not to be construed as limiting the scope thereof.

### EXAMPLES

### 1. Topical Application of C7 Promoted Wound Healing

A 1.0-cm² (1 cm×1 cm) square full-thickness excision wound was made on the mid-back of 8 to 10 week old athymic nude mice and human recombinant C7 (40 μg) was applied topically once on day 0 (n=20 mice per group).

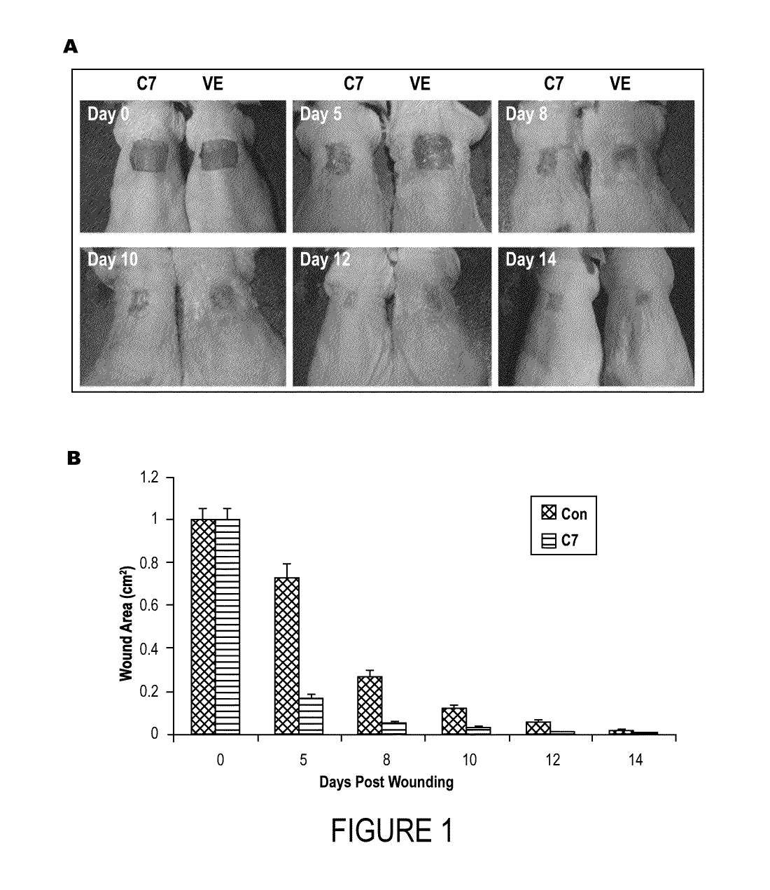

FIG. 1A shows photographs of the wound site on representative days 0, 5, 8, 10, 12 and 14. Wound sizes were significantly reduced in mice topically treated with C7, but not the vehicle cream alone (VE) (Control). FIG. 1B shows the wound size plotted against the days (mean±SD wound size measurements at day 0, 5, 8, 10, 12 and 14 post-wounding, n=20 mice for each group).

### 2. C7 Applied Topically on the Wound Incorporates into the Mice's BMZ

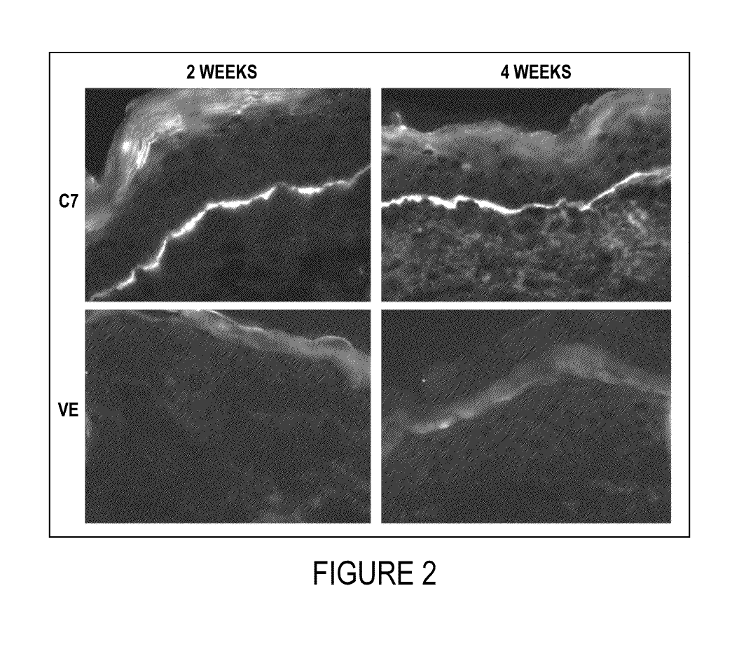

Immunofluorescence staining of the mice's skin was performed with an antibody specific for human C7 at 2 and 4 weeks after topical application of C7. Compared to the vehicle-treated wounds, the healed wounds treated with C7 demonstrated a linear pattern of C7 deposition at the BMZ. As shown in FIG. 2, we found that C7 remained localized at the BMZ up to 8 weeks after initial application.

### 3. Co-Localization of Human C7 with Mouse C7 at the Mice's BMZ

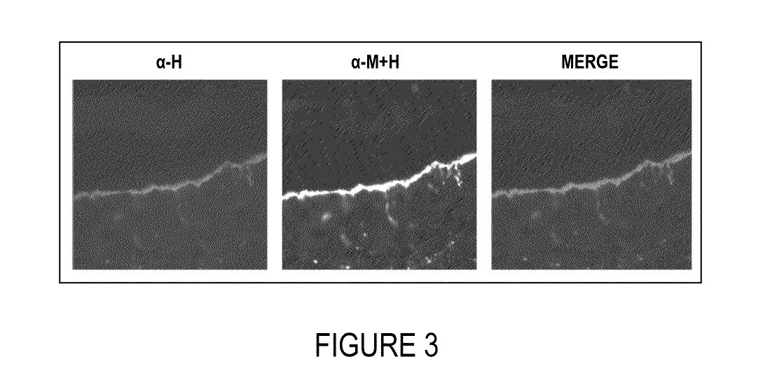

Immunofluorescence staining of mice skin was performed 2 weeks after the topical application of C7. Referring to FIG. 3, the skin sections were labeled with either a monoclonal antibody specific for human C7 (red, panel α-H) or a rabbit polyclonal antibody that recognizes both mouse and human C7 (green, panel α-M+H). Merged images demonstrate co-localization of human C7 with mouse C7 at the mouse's BMZ.

### 4. C7 Strongly Promotes Human Keratinocyte Migration

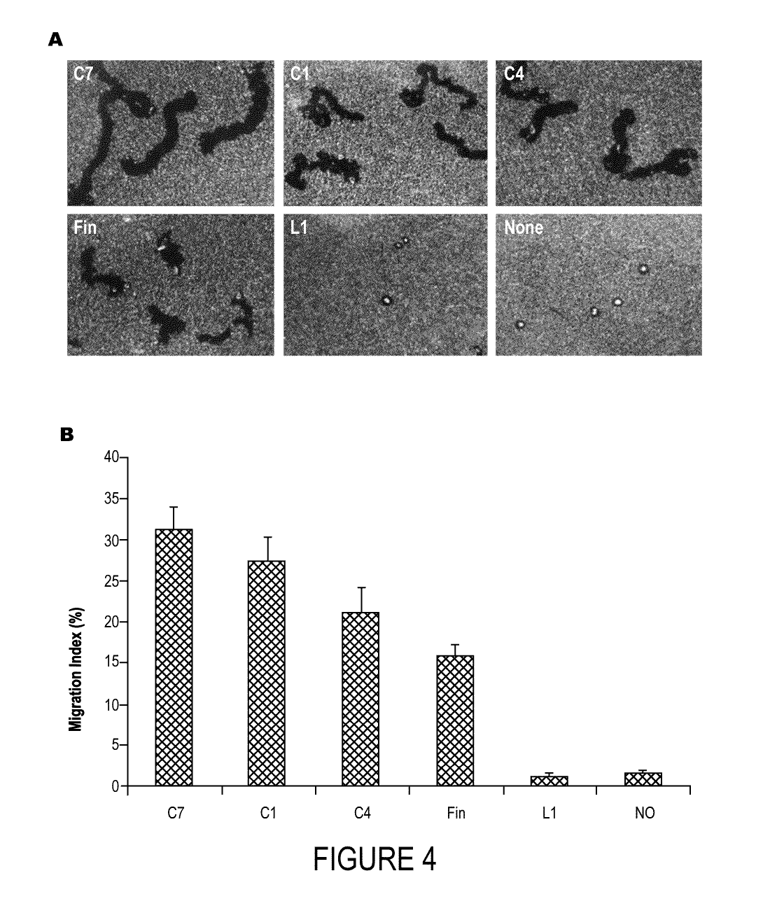

In this experiment, coverslips were coated with colloidal gold salts, then keratinocytes (KC) were plated on no matrix (None), type I collagen (30 μg/ml), type IV collagen (40 μg/ml), fibronectin (40 μg/ml), laminin 1 (80 μg/ml) or recombinant C7 (20 μg/ml) and incubated for 18 hr. Representative fields were photographed at 40× under dark field optics. The results are shown in FIG. 4A.

FIG. 4B shows the migration index expressed as the percentage of the total field area consumed by migration tracks. Error bars shows the standard error (SE) of three different experiments. Note that C7 is a potent pro-motility matrix for human keratinocytes (HKC).

### 5. Topical Application of C7 Enhances Re-Epithelialization of Skin Wounds

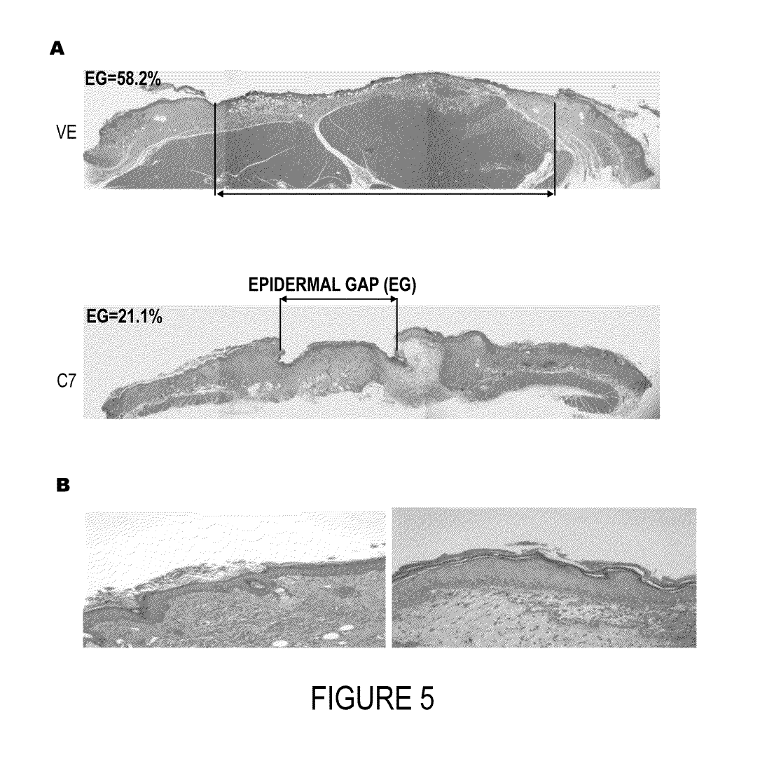

FIG. 5A shows composite pictures of hematoxylin and eosin (H&E) staining of wounds 8 days after treatment with vehicle alone (top panel) or C7 (bottom panel). Note that C7 treatment reduces epidermal gap distance.

FIG. 5B shows higher magnifications of the vehicle-treated (left) and C7-treated (right) wounds. Note that C7 treatment increases epidermal thickness and basal cell density.

### 6. Effect of C7 Domains on Migration of Human Keratinocytes

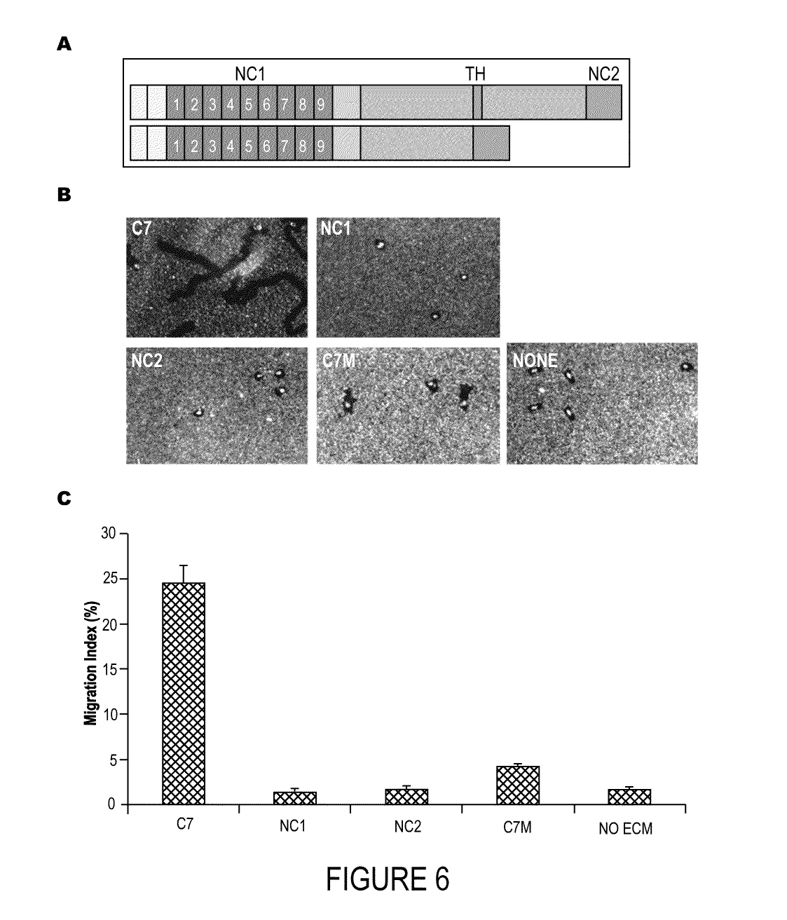

In this experiment, we investigated the role of the various C7 domains may have on migration of human keratinocytes. FIG. 6A shows a schematic view of C7 domain organization and mini-collagen C7M. C7 consists of a 2944 amino acid sequence with a central triple-helical domain (TH), flanked by a large amino-terminal non-collagenous domain, NC1, and a smaller carboxyl-terminal non-collagenous domain NC2. The TH domain contains a noncollagenous 39 amino acid hinge region. C7M is the minigene cDNA construct (C7M) which contains intact NC1 and NC2 domains and a truncated TH domain with an in-frame deletion from amino acid 1920 to 2603.

We coated coverslips with colloidal gold and plated keratinocytes on C7 (20 μg/ml), NC1 (40 μg/ml), NC2 (40 μg/ml), C7M (40 μg/ml) or no matrix and incubated for 18 hr. Representative fields were photographed at 40× under dark field optics. FIG. 6B shows the resulting photographs.

FIG. 6C shows a plot of migration index expressed as the percentage of the total field area consumed by migration tracks. Error bars represent the standard error (SE) of three duplicate experiments. Please note that a 684 helical domain that is deleted in C7M minicollagen is responsible for C7-driven motility.

### 7. Topical C7 Treatment is Superior to Topical PDGF Treatment for Wound Healing

In this experiment, we compared the wound healing enhancement power of C7 to PDGF, an FDA-approved drug for treating skin wound.

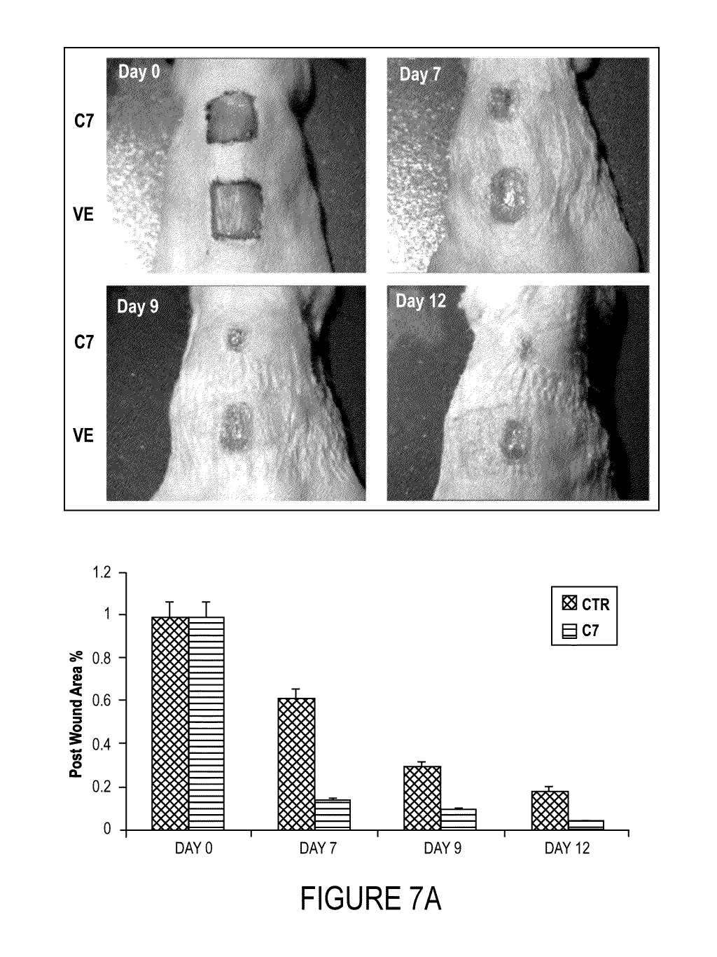

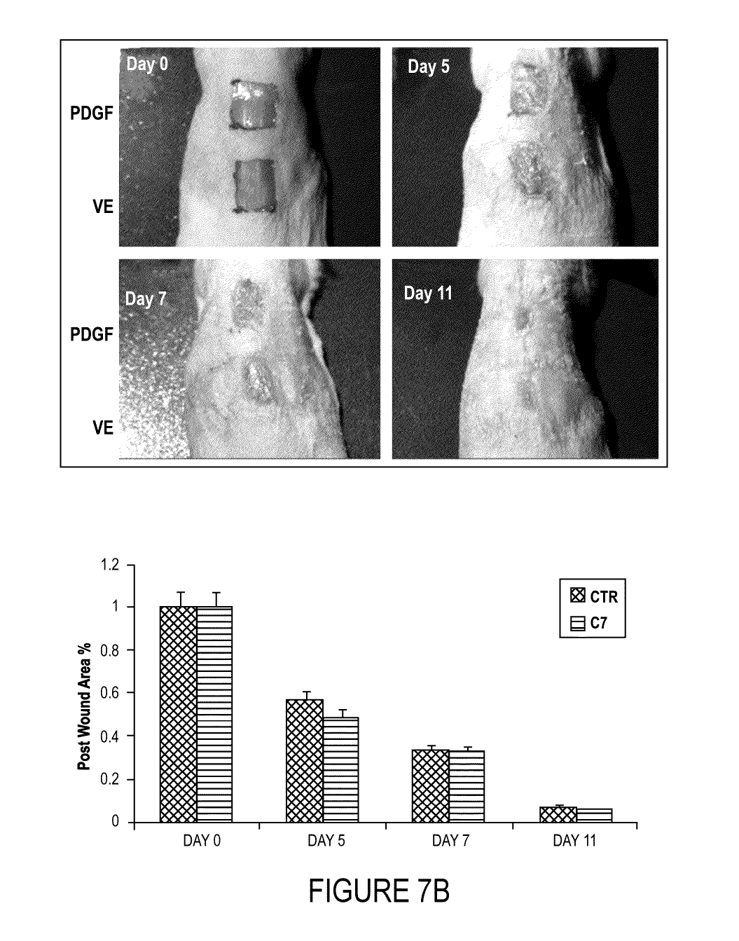

We made 1.0-cm² (1 cm×1 cm) square full-thickness excision wounds on the mid-back of 8 to 10 week old athymic nude mice and then treated the wound topically with (A) C7 (40 μg) and (B) PDGF (20 μg) once on day 0 (n=10 mice per group). The result of C7 treated wounds are shown in FIG. 7A, PDGF treated wounds are shown in FIG. 7B.

Note that compared to controls, wound sizes were significantly reduced in mice topically treated with C7, but not in mice topically treated with PDGF.

### 8. Topical C7 Treatment Enhances Dermal Remodeling of Collagen Fibers

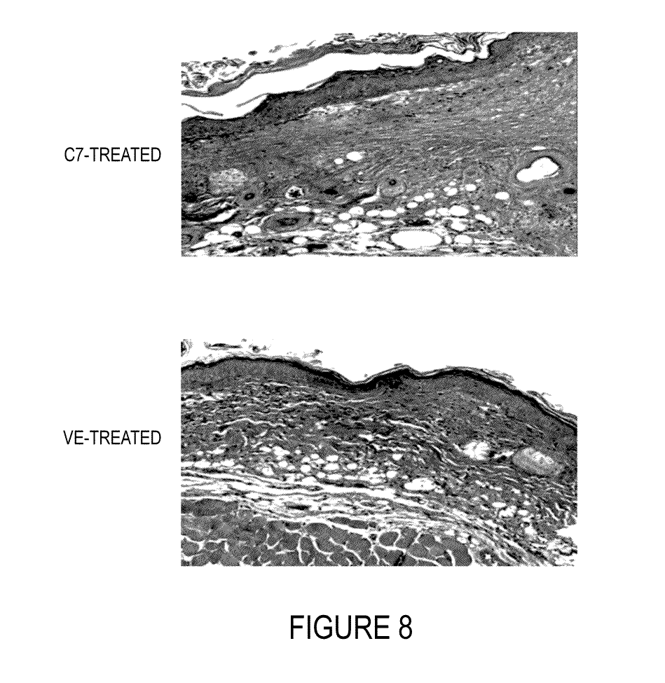

In this experiment, we examined the dermal remodeling ability of topical C7 treatment. FIG. 8 shows representative images of C7-treated and vehicle-treated 8-day wounds stained with Masson's trichrome. Note that in comparison to the vehicle-treated wound, the dermis of the C7-treated wound exhibited an increased density of neatly organized parallel collagen fibers.

### 9. Topical Treatment of C7 Down-Regulates Expression of α-SMA

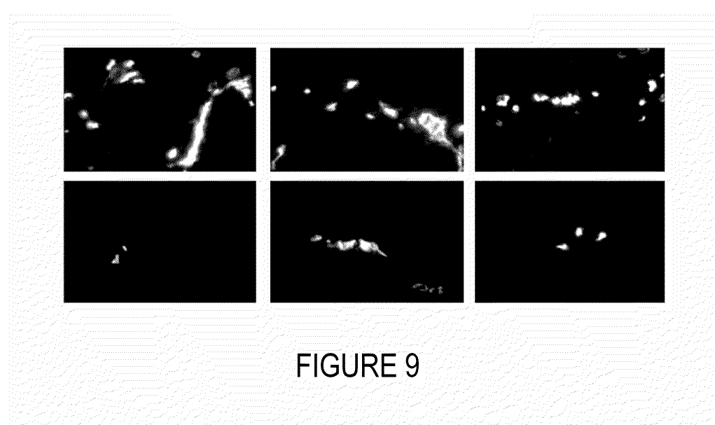

FIG. 9 shows immunofluorescence staining of the mouse's healed skin. The staining was performed with antibodies specific for smooth muscle actin 2 weeks after topical application of vehicle only (top panel) and C7 (bottom panel). Note that C7-treated skin resulted in lower expression of smooth muscle actin, a primary mediator of wound contraction.

The expression of α-SMA is known to correlate directly to wound healing, appearing at the initiation of wound healing and disappearing at the end of wound contraction process. The result of this experiment shows that topical C7-treated wound reached completion of wound healing much faster than non-treated wound.

### 10. Topical Treatment of C7 Enhances Wound Healing in Diabetic Mice

In this experiment, we further investigated the wound healing ability of topical C7-treatment in a mouse diabetes model. We made a 1.2 cm diameter circular wound on the back of db/db mice and then applied C7 (40 μg) topically in the vehicle containing 5% carboxymethylcellulose onto the wound.

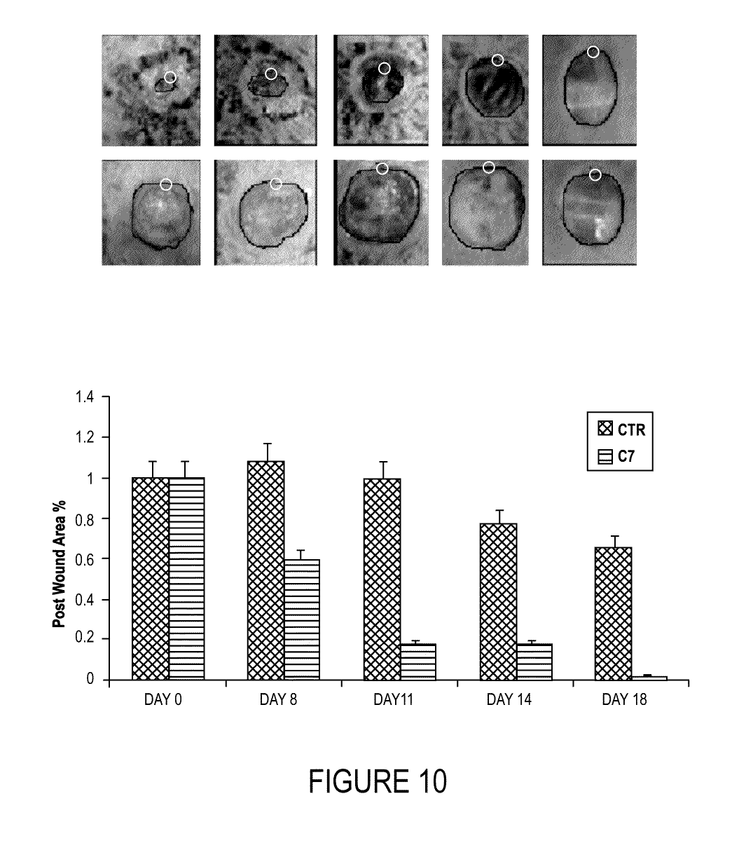

FIG. 10 shows photographs of the wound on representative days 0, 8, 11, 14 and 18 wounds (top) and their measurements (bottom).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. Those skilled in the art will recognize, or be able to ascertain using no more than routine experimentation, many equivalents to the specific embodiments of the method and compositions described herein. Such equivalents are intended to be encompassed by the following claims.

### REFERENCES

The following references are incorporated into this application by reference:
1. Stein, J Chronic wounds on the rise. Internal Medicine World Report 2000, p.
2. Hess, C T (1999). Care for a diabetic ulcer. Nursing 29: 71.
3. Brown, G L et al. (1989). Enhancement of wound healing by topical treatment with epidermal growth factor. N Engl J Med 321: 76-79.
4. Goldman, R (2004). Adv Skin Wound Care 17: 24. Review.
5. Fu, X, Li, X, Cheng, B, Chen, W and Sheng, Z (2005). Engineered growth factors and cutaneous wound healing: success and possible questions in the past 10 years. Wound Repair Regen 13: 122-130.
6. Lin, A N and Carter, D M (eds) (1992). Epidermolysis Bullosa: Basic and Clinical Aspects. Springer-Verlag Inc.: NY.
7. Briggaman, R A and Wheeler Jr, C E (1975). The epidermal-dermal junction. J Invest Dermatol 65: 71-84.
8. Uitto, J and Christiano, A M (1994). Molecular basis for the dystrophic forms of epidermolysis bullosa: mutations in the type VII collagen gene. Arch Dermatol Res 287: 16-22.
9. Burgeson, R E (1993). Type VII collagen, anchoring fibrils, and epidermolysis bullosa. J Invest Dermatol 101: 252-255.
10. Sakai, L Y, Keene, D R, Morris, N P and Burgeson, R E (1986). Type VII collagen is a major structural component of anchoring fibrils. J Cell Biol 103: 1577-1586.
11. Ortiz-Urda, S et al. (2002). Stable nonviral genetic correction of inherited human skin disease. Nat Med 8: 1166-1170.
12. Chen, M et al. (2002). Restoration of type VII collagen expression and function in dystrophic epidermolysis bullosa. Nat Genet 32: 670-675.
13. Woodley, D T et al. (2003). Normal and gene-corrected dystrophic epidermolysis bullosa fibroblasts alone can produce type VII collagen at the basement membrane zone. J Invest Dermatol 121: 1021-1028.
14. Ortiz-Urda, S, Lin, Q, Green, C L, Keene, D R, Marinkovich, M P and Khavari, P A (2003). Injection of genetically engineered fibroblasts corrects regenerated human epidermolysis bullosa skin tissue. J Clin Invest 111: 251-255.
15. Woodley, D T et al. (2004). Intradermal injection of lentiviral vectors corrects regenerated human dystrophic epidermolysis bullosa skin tissue in vivo. Mol Ther 10: 318-326.
16. Woodley, D T et al. (2004). Injection of recombinant human type VII collagen restores collagen function in dystrophic epidermolysis bullosa. Nat Med 10:693-695.
17. Singer, A J and Clark, R A F (1999). Cutaneous wound healing. Rev N Engl J Med 341:738-746.
18. Li, W, Fan, J, Chen, M and Woodley, D T (2004). Mechanisms of human skin cell motility. Histol Histopathol 19: 1311-1324.
19. Kim, Y H, Woodley, D T, Wynn, K C, Giomi, W and Bauer, E A (1992). Recessive dystrohpic bullosa phenotype is preserved in xenografts using SCID mice: development of an experimental in vivo model. J Invest Dermatol 92: 191-197.
20. Gallico III, G G, O'Connor, N E, Compton, C C, Kehinde, O and Green, H (1984). Permanent coverage of large burn wounds with autologous cultured human epithelium. N Engl J Med 311: 448-451.
21. Herzog, S R, Meyer, A, Woodley, D T and Peterson, H D (1988). Wound coverage with cultured autologous keratinocytes: use after burn wound excision, including biopsy follow up. J Trauma 28: 195-198.
22. Woodley, D T et al. (1988). Bound wound resurfaced by cultured epidermal autografts show abnormal reconstitution of anchoring fibrils. JAMA 259: 2566-2571.
23. Badiavas, E V, Abedi, M, Butmaarc, J, Falanga, J V and Quesenberry, P (2003). Participation of bone marrow derived cells in cutaneous wounding healing. J Cell Physiol 196: 245-250.
24. Badiavas, E V and Falanga, V (2003). Treatment of chronic wounds with bone marrow-derived cells. Arch Dermatol 139: 510-516.
25. Fathke, C et al. (2004). Contribution of bone marrow-derived cells to skin: collagen deposition and wound repair. Stem Cells 22: 812-822.
26. Ito, M et al. (2005). Stem cells in the hair follicle bulge contribute to wound repair but not to homeostasis of the epidermis. Nat Med 11: 1351-1354.
27. Chen, M, O'Toole, E A, Li, Y Y and Woodley, D T (1999). a2b1 integrin mediates dermal fibroblast attachment to type VII collagen via a 158 amino acid segment of the NC1 domain. Exp Cell Res 249: 231-239.
28. Haapasalmi, K et al. (1995). Expression of epithelial adhesion proteins and integrins in chronic inflammation. Am J Pathol 147: 193-206.
29. Woodley, D T, Peterson, H D and Herzog, S R. (1988). Burn wounds resurfaced by cultured epidermal autografts show abnormal reconstitution of anchoring fibrils. JAMA 259: 2566-2571.
30. Chen, M et al. (1997). Interactions of the amino-terminal noncollagenous (NC1) domain of type VII collagen with extracellular matrix components. J Biol Chem 272: 14516-14522.
31. Chen, M, Marinkovich, M P, Jones, J J, O'Toole, E A and Woodley, D T. (1999). NC1 domain of type VII collagen binds to the beta 3 chain of laminin 5 via a unique subdomain within the fibronectin-like repeats. J Invest Dermatol 112: 177-183.
32. Rousselle, P, Keene, D R, Ruggiero, F, Champliaud, M F, Rest, M and Burgeson, R E. (1997). Laminin 5 binds the NC-1 domain of type VII collagen. J Cell Biol 138: 719-728.
33. Hopkinson, I, Anglin, I E, Evans, D L and Harding, K G (1997). Collagen VII expression in human chronic wounds and scars. J Pathol 182: 192-196.
34. Chen, M, Petersen, M J, Li, H-L, Cai, X-Y, O'Toole, E A and Woodley, D T (1997). Ultraviolet A irradiation upregulates type VII collagen expression in human dermal fibroblasts. J Invest Dermatol 108: 125-128.
35. Gammon, W R, Briggaman, R A, Inman III, A Q, Queen, L L and Wheeler, C E (1984). Differentiating anti-lamina lucida and anti-sublamina densa anti-BMZ antibodies by indirect immunofluorescence on 1.0M sodium chloride-separated skin. J Invest Dermatol 82: 139-144.
36. Keene, D R, Sakai, L Y, Lunstrum, G P, Morris, N P and Burgeson, R E (1987). Type VII collagen forms an extended network of anchoring fibrils. J Cell Bio 104: 611-621.
37. Champliaud, M F, Lunstrum, G P, Rousselle, P, Nishiyama, T, Keene, D R and Burgeson, R E (1996). Human amnion contains a novel laminin variant, laminin 7, which like laminin 6, covalently associates with laminin 5 to promote stable epithelial-stromal attachment. J Cell Biol 132: 1189-1198

1-13. (canceled)

14. A method of restoring Collagen VII (C7) in a basement membrane zone of a subject in need thereof, comprising:
topically applying an effective amount of a wound healing pharmaceutical composition directly to a wound site of a skin of the subject, wherein said pharmaceutical composition comprises C7 and a pharmaceutically acceptable carrier, thereby restoring C7 in the basement membrane zone.

15. The method of claim 14, wherein the subject has a Dystrophic form of Epidermolysis Bullosa (DEB).

16. The method of claim 15, wherein the DEB is recessive DEB (RDEB).

17. The method of claim 14, wherein the C7 remains in the basement membrane zone for up to 8 weeks after a single administration.

18. The method of claim 14, wherein the method further comprises determining an increase in C7 in the basement membrane zone of the wound site after administration of the pharmaceutical composition in comparison to an amount of C7 in a basement membrane zone of a wound site that has not been treated with the pharmaceutical composition.

19. The method of claim 14, wherein the subject is a human.

20. The method of claim 14, wherein the pharmaceutically acceptable carrier is 10% carboxymethylcellulose salt gel.

21. The method of claim 14, wherein the pharmaceutical composition further comprises Hsp90.

22. The method of claim 14, wherein said pharmaceutically acceptable carrier is capable of encapsulating the skin wound enhancer for extended time release.

23. The method of claim 14, further comprising applying Platelet Derived Growth Factor (PDGF) to the wound site.

24. A method of promoting re-epithelialization in a subject in need thereof comprising:
topically applying an effective amount of a wound healing pharmaceutical composition to a skin of the subject,
wherein said pharmaceutical composition comprises C7 and a pharmaceutically acceptable carrier,
wherein an epidermal gap distance of the wound site is reduced in comparison to an epidermal gap distance of a wound site that has not been treated with the pharmaceutical composition.

25. The method of claim 24, wherein a keratinocyte migration in the wound site after administration of the pharmaceutical composition is greater than a keratinocyte migration in a wound site that was not treated with the pharmaceutical composition.

26. The method of claim 24, wherein the subject is a human.

27. The method of claim 24, wherein the subject is suffering from a condition selected from the group consisting of DEB, RDEB and diabetes

28. The method of claim 24, further comprising applying PDGF to a wound site of the skin.

29. A pharmaceutical composition comprising:
an effective amount of a skin wound healing enhancer; and
a pharmaceutically acceptable carrier, wherein said skin wound healing enhancer is selected from the group consisting of C7, mini-C7, a variant thereof and a combination thereof, and wherein said pharmaceutically acceptable carrier comprises 10% carboxymethylcellulose salt gel.

30. The pharmaceutical composition of claim 29 further comprising a protein stabilizing agent.

31. The pharmaceutical composition of claim 30, wherein the protein stabilizing agent is Hsp90.

32. The pharmaceutical composition of claim 29, wherein said pharmaceutically acceptable carrier is capable of encapsulating the skin wound healing enhancer for extended time release.

33. The pharmaceutical composition of claim 29, further comprising PDGF.
